(19) United States
(12) Patent Application Publication
(10) Pub. No.: US 2024/0166981 A1
(43) Pub. Date: May 23, 2024

(54) MICROBIOLOGICAL TESTING DEVICE, MICROBIOLOGICAL TESTING ASSEMBLY AND CAP FOR A MICROBIOLOGICAL TESTING DEVICE

(71) Applicant: Tintometer GmbH, Dortmund (DE)

(72) Inventors: James P. GOODMAN, Fort Collins, CO (US); Elmar GRABERT, Dortmund (DE)

(21) Appl. No.: 18/057,257

(22) Filed: Nov. 21, 2022

(51) Int. Cl. C12M 1/12; C12M 1/00; C12M 1/24; C12Q 1/04

(52) U.S. Cl. CPC C12M 25/04; C12M 23/08; C12M 23/38; C12Q 1/04

(57) ABSTRACT

A microbiological testing device (10), comprises a growth medium support plate (12) and a cap (14) attached to the growth medium support plate (12), the growth medium support plate (12) comprising a microbiological testing area (60), wherein the microbiological testing area (60) comprises at least one recess (66) for receiving a microbiological growth medium (62), wherein the least one recess (66) comprises a bottom surface (68) and at least one anchoring element (74) protruding from the bottom surface (68), and wherein the at least one anchoring element (74) comprises an undercut (76) for engaging with the microbiological growth medium (62). Further, a microbiological testing assembly and a cap for the microbiological testing device are described.

### FIELD OF THE INVENTION

The present invention relates to a microbiological testing device, a microbiological testing assembly comprising the microbiological testing device, and a cap for the microbiological testing device.

In order to examine the presence of microorganisms in liquids or on solid surfaces, sample material has to be collected prior to analysis. The step of collecting sample material is often critical since the amount of collected material is decisive for the sensitivity of the later analysis method. Usually, samples of a liquid are collected by taking an amount of liquid, while samples from solid surfaces are collected by wiping off the respective surface using a cotton swab or similar tools. In both cases, the collected samples are transferred onto a culture medium in a petri dish in a laboratory setting. The culture medium in the petri-dish is incubated for a predetermined amount of time. After incubation, microorganisms in the form of colonies appear on the culture medium allowing a qualitative and quantitative analysis depending on the culture medium used.

One drawback of the above process is that it is often necessary to collect samples from remote sources and to transfer the collected samples subsequently to a laboratory environment for detailed analysis. Often it is also required that the sample collection is carried out by skilled personnel since any faults in sample collection can be detrimental for the later analysis. In order to avoid these drawbacks, microbiological test devices have been developed that allow a fast and reliable sample collection by untrained personnel. One of these microbiological testing devices are dipslides.

Dipslides are known in the art as convenient to use, rapid tests, for evaluating the presence of microorganisms in liquids or on solid surfaces. A dipslide consists of a paddle-like plastic support attached to a cap, wherein the paddle-like plastic support is coated with a culture medium or growth medium for the growth and detection of microorganisms.

Dipslides are ready-to-use devices, hence they typically come with a vial for encapsulating the paddle-like plastic support prior to analysis.

For the collection of sample material, the paddle-like plastic support is removed from the vial and the culture medium of the paddle-like plastic support is pressed either on a solid surface or dipped into a liquid. After collecting the sample material, the paddle-like plastic support with the coated culture medium is again encapsulated in the vial for transportation purposes. Subsequently, the dipslides are incubated at a temperature in the range of 30-35° C. in a specialized incubator allowing the growth of microorganisms on the culture medium. Typically, the incubator is specifically made for the type of dipslides used, such that no specialized laboratory is required. After a certain amount of incubation time, microorganisms in the form of colonies appear on the growth medium allowing a quantitative analysis by simply counting the number of colonies. Qualitative analysis can also be carried out depending on the culture medium used.

In general, dipslides indicate the presence of microorganisms, for example fungi or bacteria, in a variety of applications. These include applications related to, but not limited to, industrial water, industrial fluids, food manufacturing, dental practices, breweries, environmental hygiene, leather industry, fuels, dairy industry, pools & spas and cosmetics.

Dipslides are widely known in the art. For example, dipslides are disclosed in US 2010/0 079 751 A1, US 2006/0 121 601 A1 and U.S. Pat. No. 4,865,988 A.

In known dipslides, the growth medium is merely cast into a recess or a pan-like structure on the plastic support. Thus, the growth medium is only loosely bonded to the plastic support. As a result, the growth medium needs to be handled with care, such that the culture medium is not damaged or scratched off during sample collection.

US 2006/0 121 601 A1 discloses a growth medium support plate comprising of a body part and a stem member, wherein the stem member is situated essentially in the same plane with the body part and extending therefrom, for connecting a cap to said support plate. The stem member is connected to the support plate so as to be flexible in regard to the plane of the support plate. The stem member is connected to the support plate body part in a flexurally stiff fashion. The support plate itself comprises a narrow growth medium side portion having a smooth surface or contoured surface with bumps or dimples in order to increase the surface area for improving the adhesion of the growth medium. There is no disclosure on how the bumps or dimples are shaped or crafted.

However, commonly available dipslides still suffer from fading adhesion of the culture medium to the surface of the support plate over time. Typically, the culture medium is composed of a gel-like substance such as agar and therefore shrinks over time due to evaporation of moisture. Shrinkage can weaken the adhesion of the culture medium to the surface of the support plate and, in the worst case, lead to a detachment of the agar medium from the support plate. This phenomenon is often observed during long-term storage and/or shipping of the dipslides and can reduce shelf life of the dipslides significantly.

In addition, loss of adhesion of the culture medium may occur in freeze and thaw cycles, during sample handling, or by means of shock and vibrational impacts during manufacturing and transport.

So far, these issues have not been addressed.

### SUMMARY OF THE INVENTION

Thus, it is an objective of the present invention to provide a microbiological testing device that overcomes one or more of the above-mentioned disadvantages of the prior art.

It is a further objective of the present invention to provide a low-cost microbiological testing assembly, which enables secure and easy handling.

Another objective of the present invention is to provide a cap for a microbiological testing device, which allows for an improved handling of a microbiological testing assembly equipped with said cap and for an improved storage and packing of a plurality of microbiological testing assemblies.

In a first aspect, the invention relates to a microbiological testing device comprising a growth medium support plate and a cap attached to the growth medium support plate. The growth medium support plate comprises a microbiological testing area extending between a distal portion having a free end and a proximal portion attached to the cap, wherein the microbiological testing area comprises at least one recess for receiving a microbiological growth medium. The at least one recess comprises a bottom surface and at least one anchoring element protruding from the bottom surface. The at least one anchoring element comprises an undercut for engaging with the microbiological growth medium.

Use of the at least one anchoring element comprising an undercut allows that the at least one anchoring element protruding from the bottom surface engages with the growth medium such that the growth medium is firmly adhered to the microbiological testing area. In other words, the bond strength between the culture medium and the microbiological testing area is increased.

The undercut of the at least one anchoring element provides improved adhesion and physical engagement with the culture medium over time. When encountering shrinkage of the culture medium over time, the at least one anchoring element provides a locking interaction between the undercut and the culture medium, and is able to firmly anchor the culture medium to the bottom surface of the support plate. As a result, the culture medium cannot detach from the support plate, although other parts of the culture medium may shrink and detach from circumferential edges of the plastic support. In general, an improved durability and shelf life of the dipslide is obtained.

Moreover, the undercut of the at least one anchoring element offers the advantage that the agar medium can have improved mechanical attachment to the support plate. Thus, the dipslides may allow for improved handling since the agar medium will not be easily scratched off the growth medium support plate, or easily washed off, during sample collection.

According to a preferred embodiment, the at least one anchoring element is inclined with respect to a plane perpendicular to the bottom surface to form the undercut between the bottom surface and the at least one anchoring element.

In principle, the undercut is formed by the inclined anchoring element. Thus, one part of growth medium is sandwiched between the bottom surface of the support plate and the undercut of the anchoring element. Thus, the part of the growth medium in between is adhered to the anchoring element, which results in an improvement of the overall adhesion of the growth medium to the bottom surface of the support plate. This effect is mainly due to the fact that the undercut of the anchoring element provides a counter to the frictional forces that can be applied to the growth medium during sample collection.

According to another preferred embodiment, an angle between an inclined direction of the at least one anchoring element and the plane perpendicular to the bottom surface is in the range of 5 to 45°.

It has been found that an anchoring element with an angle between an inclined direction of the at least one anchoring element and the plane perpendicular to the bottom surface in the range of 5 to 45° can be produced fast and easy by an injection molding process without use of advanced tooling equipment.

In principle, the at least one anchoring element is not limited to a particular shape. Rather, the at least one anchoring element can have any possible shape if it has at least one undercut relative to the bottom surface of the recess for receiving the microbiological growth medium.

According to a further preferred embodiment, the at least one anchoring element has a free end, wherein the free end is tapered. A tapered free end of the at least one anchoring element is generally advantageous in terms of rapid and cost-effective fabrication of the anchoring element. In particular, it was found that an anchoring element having a tapered free end can be removed from the injection mold without using advanced and expensive tooling equipment.

According to another preferred embodiment, the at least one anchoring element is selected from the group consisting of pins, fins, ribs, rods, cones, ridges, polyhedrons and plates. More preferably, the at least one anchoring element is in the form of pins, fins, ribs or plates.

It is preferred that the at least one anchoring element is selected from the above group and has a free end, which is tapered. In another embodiment, the growth medium support plate comprises a plurality of anchoring elements arranged on the bottom surface of the recess for receiving the microbiological growth medium.

Advantageously, the growth medium support plate, in particular the bottom surface of the recess, comprises more than one anchoring element. A plurality of anchoring elements can engage with more parts of the growth medium allowing an improved adhesion to the bottom surface. Preferably, the plurality of anchoring elements are arranged to provide opposing undercuts so as to increase the mechanical interlock between the anchoring element and the growth medium.

According to a further preferred embodiment, the plurality of anchoring elements forms at least one anchoring unit on the bottom surface. For example, the at least one anchoring unit can comprise 2, 3, 4, 5, 6 or more than 6 anchoring elements.

The advantage of arranging the plurality of anchoring elements in anchoring units of up to six or more anchoring elements is that the density of the anchoring elements can be locally increased over the entire bottom surface of the recess. In addition to an increased surface contact, the plurality of anchoring elements have opposing undercuts that can provide an interlocking configuration. In principle, the arrangement of anchoring elements within one anchoring unit can be arbitrary. Thus, the anchoring elements can be arranged symmetrically or randomly with respect to each other.

According to a preferred embodiment, the undercuts of the anchoring elements within an anchoring unit are each arranged in opposite or in opposing orientation to each other. This arrangement of undercuts provides a strong physical lock such that the growth medium located adjacent to an anchoring unit is firmly adhered to the bottom surface of the recess.

According to a further preferred embodiment, the anchoring elements of one anchoring unit are symmetrically arranged with respect to each other. By the virtue of providing symmetrically arranged anchoring elements in an anchoring unit, as opposed to randomly arranged anchoring elements, the adhesion of the growth medium across the bottom surface is equal in more than one direction across the bottom surface plane. In principle, the undercut is oriented along an extension direction of the anchoring element. Thus, the undercut has an orientation relative to the bottom surface. Providing a symmetrical arrangement of a plurality of oriented anchoring elements in an anchoring unit can also distribute the adhesion forces symmetrically across the plane of the bottom surface. As a result, the growth medium is homogenously adhered to the bottom surface.

In the above described embodiments, improved attachment of the culture medium to the support plate is provided by an increased surface contact, which is enhanced by the undercut of the at least one anchoring element. Thus, the undercut is arranged to provide a mechanical interlocking interaction between the support plate and the culture media. Preferably, the mechanical interlock can be achieved by providing an opposing fixed and raised feature in proximity to the undercut such as a further opposing anchoring element or a wall feature limiting lateral movement of the culture medium.

According to a further aspect of the invention, the microbiological testing area of the growth medium support plate comprises at least one microbiological growth medium located in the at least one recess.

According to a further preferred embodiment, the microbiological testing area comprises a plurality of recesses, wherein each recess of the plurality of recesses has a regular-polygonal shape that is defined by wall elements framing each of the plurality of recesses, wherein each of the plurality of recesses includes at least one anchoring unit.

Thus, a segmentation of the microbiological testing area into a plurality of sub-areas is achieved. Each of the plurality of sub-areas is separated from the neighboring sub-areas by wall elements. This allows a fast visualization and counting of the colonies formed on the growth medium after incubation of the growth medium. Furthermore, the wall elements further support the adhesion of the growth medium to the microbiological testing area. While the undercut protects the growth medium from vertical frictional forces, the wall elements can protect the growth medium against horizontal frictional forces, where “horizontal” and “vertical” is defined with respect to the plane of the bottom surface.

The wall elements can also be configured as an opposing feature to the at least one anchoring element such that the culture medium is interlocked between the anchoring element and the wall element. In this interlocking configuration, the culture medium is protected against lateral frictional forces over the shelf life of the device.

According to another aspect of the invention, the microbiological testing area comprises a plurality of discrete fields, each field being composed of culture medium, wherein each discrete field of each of the plurality of discrete fields is disposed in each recess of the plurality of recesses.

According to another aspect of the invention, at least two of the plurality of discrete fields have a different formulation of the culture medium. It might also be that each plurality of discrete fields of culture media has a formulation being different from the formulations of each of the other plurality of discrete fields.

In particular, agar is used as a culture medium. In this respect, the term “formulation” refers to a chemical composition of the agar used in the device, wherein the agar is configured to detect one sort of microorganism. Different formulations of culture medium can therefore detect different sorts of microorganisms, which allow for performing more types of testing by one single dipslide device. Multiple agar media on one side of the paddle substrate enables fast and easy visualization between different test results relative to each agar media.

Providing a plurality of different discrete fields of culture medium, with each field having its own formulation with respect of the culture medium, offers the advantage that a variety of different microorganisms could be detected by merely using a single dipslide.

According to a further preferred embodiment, the cap and the growth medium support plate are made as one-piece, preferably wherein said one-piece is injection molded from a thermoplastic polymer selected from the group consisting of high-density polyethylene and low-density polyethylene and combinations thereof.

Thus, the microbiological testing device comprising the cap and the support plate can be made in a one-step production. In principle, any thermoplastic material can be used. However, high-density polyethylene (HDPE), linear low-density polyethylene (LLDPE), low-density polyethylene (LDPE) and polypropylene are preferred thermoplastic materials since they are widely available at low cost and can be handled well in an injection molding process. In addition, HDPE, LLDPE and LDPE are sufficiently stable to be used as the hinge in the connection portion. Thus, no mechanical hinge needs to be incorporated inside the microbiological testing device.

According to a further aspect, the invention relates to a microbiological testing assembly comprising a microbiological testing device and a transparent vial for receiving the microbiological testing device. The microbiological testing device comprises a growth medium support plate and a cap attached to the growth medium support plate. The growth medium support plate comprises a microbiological testing area extending between a distal portion having a free end and a proximal portion attached to the cap. The vial comprises an open end, a closed end and an interior located in-between, said open end comprises an opening that is adapted to receive the growth medium support plate such that the growth medium support plate can be completely encapsulated in the interior of the vial. The cap comprises at least one bayonet fastening adapted to engage with the open end of the vial for fastening the vial to the cap.

Accordingly, the microbiological testing assembly includes a fastening mechanism, which achieves a convenient and secure way of encapsulating the microbiological testing device in the vial after or prior to sample material collection. The bayonet fastening permits an overall improved handling of the microbiological testing assembly as compared to state-of-the-art microbiological testing assemblies, which either have no fastening mechanisms at all or an inconvenient to use fastening mechanism such as a screw cap.

Locking of the bayonet fastening also prevents unintended separation of the growth medium support plate from the vial. Known dipslides equipped with a screw cap are susceptible for mechanical impacts or vibrations, which may lead to loosening of the cap connection or an unintended opening of the dipslide. For example, the dipslides may be exposed to mechanical impacts, vibrations or pressure changes over a long period during transportation: e.g., shipping by truck, rail, sea or air. Loosened or open caps should be avoided since they can lead to contamination of the culture medium making the dipslide device unusable for the end-user.

Due to the bayonet fastening, the microbiological testing device can be seated inside the vial with a single hand motion by twisting the microbiological testing device and the vial against each other. The bayonet fastening can also be opened in the same fashion. Further, the bayonet fastening can be designed to allow aerobic growth of the microorganisms on the growth medium encapsulated inside the interior of the vial by providing a non-hermetic seal of the vial from the environment.

The microbiological testing device can have the features as described above.

In a preferred embodiment, the bayonet fastening comprises at least one receptor opening which extends through an abutment portion of the cap in an axial direction, wherein the bayonet fastening further comprises a slot portion that extends from the receptor opening to a bearing portion in the circumferential direction of the cap.

In another embodiment, the slot portion comprises a bayonet rail that has an inclination in axial direction and a bayonet-latching element located adjacent to the bearing portion. The vial comprises at least one pin located adjacent to the opening of the vial. The at least one pin comprises a bayonet interlocking element. The at least one pin is adapted to match into the receptor opening of the bayonet fastening and to slide radially along the bayonet rail of the slot portion into the bearing portion. When reaching an end position, the pin in the bearing portion is locked by the bayonet-latching element.

When the pin is located in the bearing portion, the microbiological testing assembly is positioned in a locked state. In this state, the bayonet-latching element prevents the microbiological testing assembly from being opened accidentally, for example due to shock or vibration impact or pressure changes inside the vial. A certain amount of force needs to be applied in order to slide the bayonet interlocking element of the pin back over the bayonet-latching element to unlock the microbiological testing assembly. The bayonet interlocking element and bayonet-catching element together serve as a security feature in order to prevent unintended opening of the microbiological testing assembly.

According to a further aspect, the invention further relates to a cap for a microbiological testing device, and in particular the microbiological testing device described above. The cap comprises a disk-shaped bottom plate, having a bottom side that is attachable to a growth medium support plate, and an abutment portion located opposite to the bottom side and extending radially outward from the disk-shaped bottom plate. An outer diameter of the abutment portion is greater than an outer diameter of the disk-shaped bottom plate, wherein a shoulder is formed between the disk-shaped bottom plate and the abutment portion.

On the front side of the cap, opposite to the bottom side of the disk-shaped bottom plate, the disk-shaped bottom plate and the abutment surface form a trough. The trough thus defines a recess surrounded by a circumferential side wall.

The cap according to the invention improves handling of the microbiological testing device. The different outer diameters of the disk-shaped bottom plate and the abutment portion hinder direct contact of the microbiological testing assemblies when the microbiological testing assemblies are closely packed for storage or shipping by the end user. In particular, a direct contact of the microbiological testing assemblies is prevented since the offset of the abutment portion maintains separation between adjacent vials. Thus, abrasion of the vials due to shock and vibration during handling is prevented. Abrasion would reduce the clarity and the optical transparency of the vial. However, a clear vial is required such that a user can see whether there are any bacteria and or fungi growth on the growth medium encapsulated in the vial.

In a further embodiment, an outer diameter of the disk-shaped bottom plate and/or the circumferential side wall tapers from the abutment portion towards the bottom side.

The tapering of the disk-shaped bottom plate or circumferential side wall towards the bottom side enables an improved engagement of the opening of the vial with the disk-shaped bottom plate so that the disk-shaped bottom plate can slide smoothly into the opening of the vial.

In a further embodiment, the trough has a bottom on the front side of the cap configured as a writable surface.

A writable surface is of advantage for the handling and usage of the microbiological testing device. For example, the date, the sample number, the place of sample collection or other experimentally relevant data can be labelled onto the writable surface such that the data can be directly associated with the respective microbiologic testing device. Therefore, it is no longer necessary to note the respective data with other means such as a separate lab book. Furthermore, the common practice of writing relevant data directly onto the vial can be avoided, which also comes along with certain drawbacks such as the smearing of the writing during handling the vials. The writable surface can be labeled with a commonly available marker pen.

In another embodiment, the writable surface is circumferentially framed by the circumferential side wall of the trough.

The side wall of the trough framing the writable surface prevents the writing or labelling on the writable surface from being wiped off or smeared during handling the microbiological testing device.

According to a further aspect of the invention, the cap is attached to a growth medium support plate forming a microbiological testing device, the growth medium support plate comprising a microbiological testing area extending between a distal portion having a free end and a proximal portion attached to the cap.

The microbiological testing device can have one or more features as described above.

According to a preferred embodiment, the cap and the growth medium support plate of the microbiological testing device are made as one-piece. More preferably, the microbiological testing device comprising the cap and the growth medium support plate is injection molded in one piece from a thermoplastic polymer.

A one-piece microbiological testing device having a cap comprising a writable surface allows for easy traceability of the dipslide. Once the cap is labeled, the labeling is directly linked to the growth medium support plate used to perform the tests for detecting microorganisms. Thus, the cap cannot get lost during sample handling. Thus, use of the one-piece microbiological testing device prevents sample inter-mixing when using multiple dipslides, which is a known issue in sample handling. In addition, the one-piece microbiological testing device is more cost effective than current two-part designs consisting of two separate injection molded parts, wherein each of the parts may require separate molding operations, The two-part designs may cause additional costs in tooling, as well as costs for the inventory management of two parts, the cost of assembly and the additional cost of quality control to ensure proper assembly integrity.

Further modifications are possible. For example, the abutment portion of the cap can comprise the bayonet fastening described above.

According to a further preferred embodiment, the cap comprises a circumferential outer face having alternately arranged convex and concave curved sections.

The alternately arranged convex and concave curved sections also can be provided in the cap described above. In particular, the circumferential outer face having convex and concave curved sections can extend from the abutment portion in axial direction towards the front side of the cap.

Providing a cap comprising alternately arranged convex and concave curved sections allows for an improved storage and handling of a microbiological testing device. In particular, the cap allows that a plurality of microbiological testing assemblies, each comprising a microbiological testing device and said cap, can be nested with an increased package density. In detail, the microbiological testing assemblies can be arranged in a plane of a quasi-close packing of equal spheres. Thus, less packaging is needed and transportation costs are reduced. Moreover, since the cap has alternately arranged convex and concave curved sections, these sections provide a better grip and accordingly a safer handling of said cap. This is mainly because the user can engage their fingers better into the curved concave sections of the cap such that grip is improved compared to the curved circular slick caps known in the art.

In a further embodiment, the circumferential outer face comprises three convex curved sections and three concave curved sections such that the cap seen from an axial direction has a quasi-hexagonal shape.

The three concave sections can engage with up to three convex sections of up to three adjacent caps each having three convex sections and three concave sections. Furthermore, convex and concave curved sections provide the cap with non-twisting properties such that the cap, and also the associated microbiologic testing device, is not able to rotate around its longitudinal axis when the microbiological testing devices are nested together. Avoiding twisting of the microbiological testing assemblies prevents abrasion of the vials such that the clear view through the vial is not impaired.

In another aspect of the invention, the at least one of the alternately arranged convex and concave curved sections comprises at least one anti-twist element, wherein the at least one anti-twist element extends from at least one of the alternately arranged convex and concave curved sections radially outwards.

The anti-twisting properties of the cap can even be further improved by introducing at least one anti-twist element to the alternately arranged convex and concave curved sections. The anti-twist element works as a further radial off-set in which the convex or concave curved edge can be aligned when a plurality of microbiological testing assemblies are nested together.

That allows for an improved transportation of the microbiological testing assemblies since the microbiological testing devices cannot rotate around their longitudinal axis, which may cause damage to the transparent vials.

### DESCRIPTION OF THE DRAWINGS

In the following, the invention will be described in detail by making reference to the annexed drawings, in which

FIG. 1 is a front view of a microbiological testing device according to the invention;

FIG. 2 is an isometric view of the microbiological testing device from FIG. 1;

FIG. 3 is a side view of the microbiological testing device from FIG. 1;

FIG. 4 is another front view of the microbiological testing device from FIG. 1;

FIG. 5 is a sectional view along section line A-A from FIG. 4;

FIG. 6 is a sectional view along section line B-B from FIG. 4;

FIGS. 7 to 10 are enlarged views of different anchoring elements and anchoring units protruding from a bottom surface of a microbiological testing device;

FIG. 11 is a sectional view along section line C-C from FIG. 4;

FIG. 12 is an isometric view of the microbiological testing device from FIG. 1, wherein the microbiological testing device comprises a growth medium;

FIG. 13 is an isometric view of the microbiological testing device from FIG. 1, wherein the microbiological testing device comprises a plurality of discrete fields, each being composed of culture medium;

FIG. 14 is an isometric view of a microbiological testing assembly comprising the microbiological testing device from FIG. 1 and a vial;

FIG. 15 is a top view of the vial from FIG. 14;

FIG. 16 is a sectional view along section line D-D from FIG. 14;

FIG. 17 is an isometric view of the cap of the microbiological testing device from FIG. 1;

FIG. 18 is a top view of the cap of the microbiological testing device from FIG. 1;

FIG. 19 is a view of detail B from FIG. 18;

FIG. 20 is a sectional view along section line E-E from FIG. 19;

FIG. 21 is a top view of the microbiological testing assembly comprising the microbiological testing device and the vial from FIG. 14;

FIG. 22 is a sectional view along section line F-F from FIG. 21;

FIG. 23 is a view of detail C from FIG. 22;

FIG. 24 is an isometric view of the microbiological testing assembly from FIG. 14 showing the working mechanism of a bayonet fastening;

FIG. 25 is an isometric view of the microbiological testing assembly from FIG. 14 in a locked state;

FIG. 26 is a top view of the microbiological testing assembly from FIG. 14 in an unlocked state;

FIG. 27 is a top view of the microbiological testing assembly from FIG. 14 in a locked state;

FIG. 28 is another top view of the cap of the microbiological testing device from FIG. 1 showing a quasi-hexagonal shape of said cap; and

FIG. 29 is an illustration showing a top view of a plurality of nested microbiological testing devices.

### DETAILED DESCRIPTION OF EMBODIMENTS

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the scope of the invention defined by the claims.

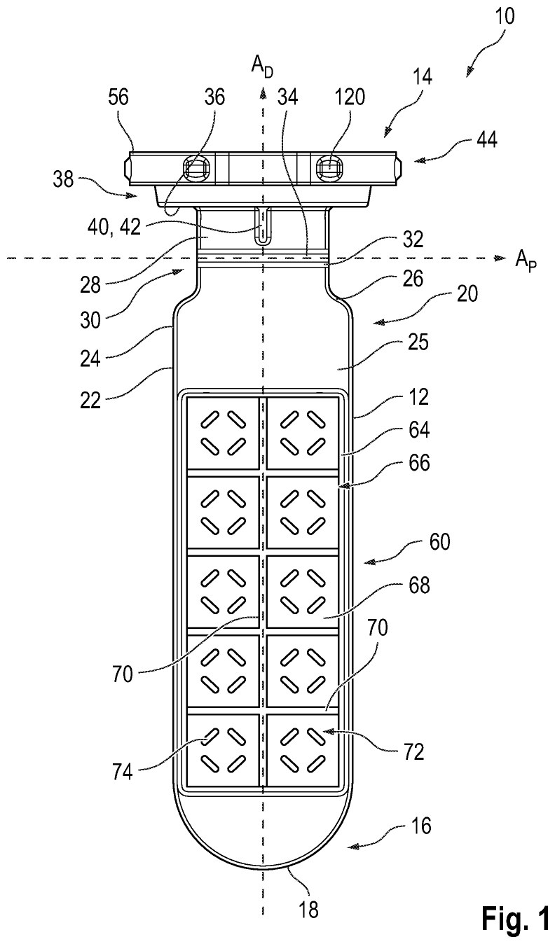

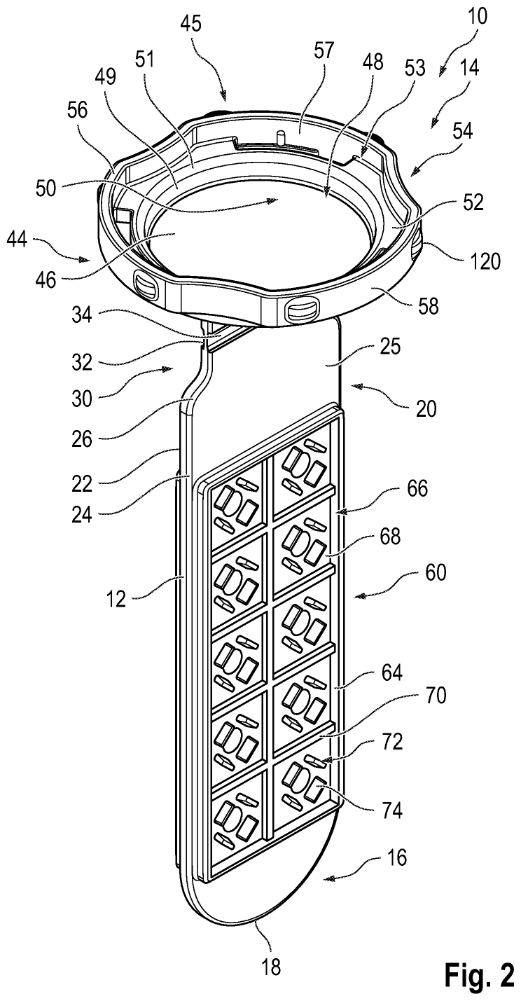

FIGS. 1 to 3 depict different views of a microbiological testing device 10.

The microbiological testing device 10 comprises a growth medium support plate 12 and a cap 14 attached to the growth medium support plate 12. Further, the growth medium support plate 12 comprises a distal portion 16 having a free end 18 and a proximal portion 20 attached to the cap 14. The growth medium support plate 12 has an elongated shape and extends along an axial direction A D from the free end 18 of the distal portion 16 to the proximal portion 20.

Further, the growth medium support plate 12 comprises two sides 22, 24 arranged opposite to each other. Both sides 22, 24 are identical to each other, i.e., they have a rotational symmetry around a central longitudinal axis of the growth medium support plate 12. In principle, each of the two sides 22, 24 define a flat and even surface 25.

A tapered portion 26 extends from the proximal portion 20 of the growth medium support plate 12 along the axial direction A D. The tapered portion 26 is a continuation of the growth medium support plate 12 and transits into a connection portion 28. The connection portion 28 is configured to attach the cap 14 to the growth medium support plate 12. The connection portion 28 is integrally connected to the growth medium support plate 12.

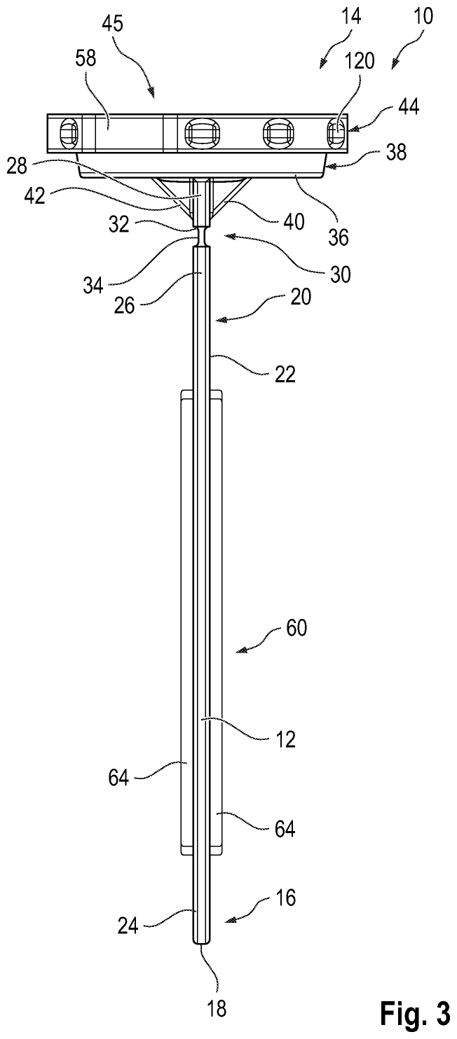

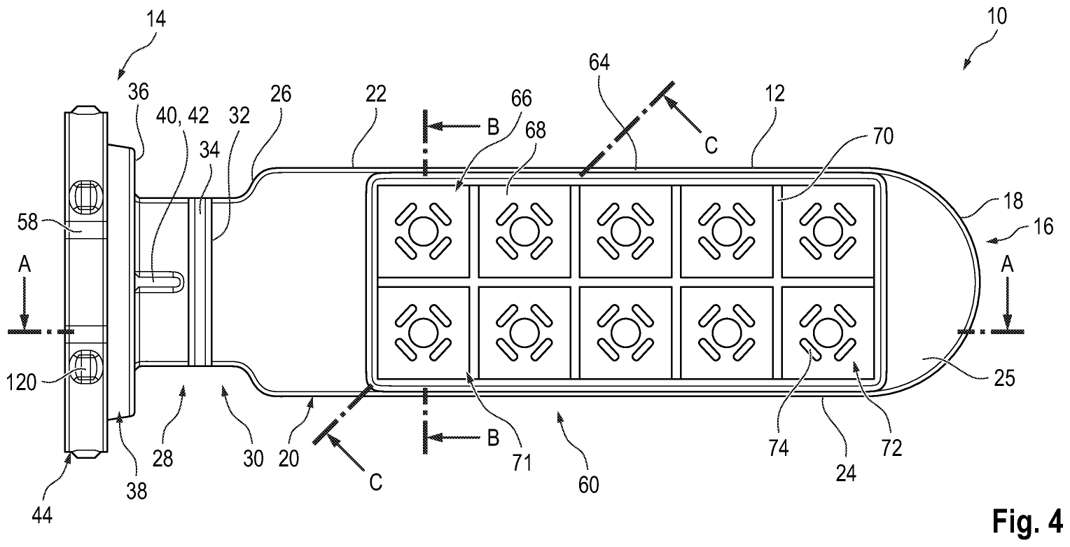

A hinge 30 is located at the connection portion 28. As shown in FIGS. 3 and 4 in detail, the hinge 30 is constructed as a weakening portion 32 having a hinge recess 34. Furthermore, the hinge 30 defines a pivot axis A_{D} running in transverse direction through the connection portion 28 and allowing to bend the growth medium support plate 12 towards the cap 14 by an angle of almost 90°. The pivot axis Aₚ is located in the plane defined by the growth medium support plate 12 and at the same time arranged perpendicular to the axial direction A_{D} of the growth medium support plate 12. In other words, the pivot axis Aₚ enables the growth medium support plate 12 to bend either the first side 22 or the second side 24 towards the cap 14.

As described above, the connection portion 28 is configured to attach the growth medium support plate 12 to the cap 14. In particular, the cap 14 comprises a disk-shaped bottom plate 38 having a bottom side 36 facing the growth medium support plate 12. The connection portion 28 is attached to the bottom side 36 of the disk-shaped bottom plate 38 of the cap 14. In particular, the connection portion 28 of the growth medium support plate 12 can be integrally connected to the disk-shaped bottom plate 38 at a center of the bottom side 36.

The cap 14 can have two support elements 40, 42, which are formed as tapered transverse bars extending in axial direction from the bottom side of the disk-shaped bottom plate 38 and abutting the connecting portion 28. The support elements 40, 42 may either define a slot for receiving the connection portion 28, or may be integrally formed with the connection portion 28 to provide a stiffening of the connection portion 28.

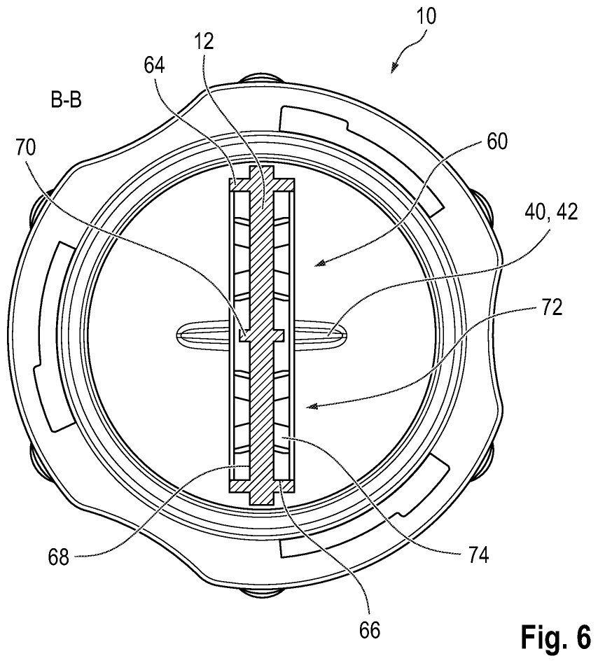

The disk-shaped bottom plate 38 has a circular shape in circumferential direction and is flat across the axial direction A D of the growth medium support plate 12. In addition, the disk-shaped bottom plate 38 has an outer diameter greater than a width of the growth medium support plate 12 in any radial direction, as shown in detail in FIG. 6. Further, the outer diameter of the disk-shaped bottom plate 38 can taper in axial direction A D towards the connection portion 28.

Furthermore, the cap 14 comprises an abutment portion 44 located opposite to the bottom side 36 of the disk-shaped bottom plate 38. In other words, the abutment portion 44 is offset from the disk-shaped bottom plate 38 on a side facing away from the growth medium support plate 12.

The abutment portion 44 also has a circular shape in circumferential direction and is flat across the axial direction A D of the growth medium support plate 12. A center of the abutment portion 44 is congruently arranged with respect to the center of the disk-shaped bottom plate 38. Accordingly, the abutment portion 44 can be described as a ring disk stacked over the disk-shaped bottom plate 38.

An outer diameter of the abutment portion 44 is greater than an outer diameter of the disk-shaped bottom plate 38. In other words, the abutment portion 44 extends radially outwards from the disk-shaped bottom plate 38. Thereby, a shoulder is formed between the disk-shaped bottom plate 38 and the abutment portion 44.

Seen from a top view perspective, the disk-shaped bottom plate 38 and the abutment portion 44 form a trough 45 defining a recess that extends from a front side of the cap 14 in axial direction A D towards the growth medium support plate 12.

In detail, the trough 45 has a bottom 46 that is formed by a top side of the disk-shaped bottom plate 38, wherein the top side is arranged opposite to the bottom side 36 of the disk-shaped bottom plate 38. The bottom 46 comprises a writable surface 48. In principle, the writable surface 48 consists of an even and flat surface being free from hinges, burns or protrusions. The writeable surface 48 may be roughened or otherwise treated to ensure adhesion of marker ink.

The writable surface 48 is circumferentially framed by a circumferential side wall 50 of the trough 45 extending in axial direction A_{D} and connected integrally to the abutment portion 44. As a result, the writable surface 48 is configured as a framed surface.

In more detail, the circumferential side wall 50 extends orthogonally from the bottom 46 of the trough 45 into an inner stage 52. The inner stage 52 features an annular shape, framing the writable surface 48 in circumferential direction. Further, the inner stage 52 can be referred to as a plateau with a flat and even plateau surface 53 arranged parallel to the writable surface 48.

The transition between the inner stage 52 and the writable surface 48 is formed by a straight wall segment 49 of the sidewall 50 of the trough 45, and an oblique wall 51 segment. Both segments 49, 51 are ring-shaped and circumferentially closed, wherein the straight wall segment 49 extends orthogonally from the writable surface 48 along the axial direction A D into the oblique wall segment 51. The oblique wall 51 segment extends radially outwards into the inner stage 52.

The inner stage 52 comprising the straight wall segment 49, the oblique wall segment 51 and the plateau surface 53 constitute the lower part of the trough 45. The plateau surface 53 corresponds to the abutment portion 44 seen from a top view of the cap 14.

The inner stage 52 of the trough 45 further transitions into a ring-shaped circumferential wall 54 that frames the plateau surface 53 of the inner stage 52. In detail, the ring-shaped circumferential wall 54 extends orthogonally from the abutment portion 44 or plateau surface 53 of the inner stage 52 in axial direction A_{D} to its free end 56. Thus, the free end 56 limits the extension of the ring-shaped circumferential wall 54 in axial direction A D forming an upper edge of the wall 54.

The ring-shaped circumferential wall 54 has two opposite sides. An inner face 57 points towards the inner stage 52 and the writable surface 48, and a circumferential outer face 58 points radially outwards. The inner face 57 is located adjacent to the inner stage 52 and confines the extension of the inner stage 52, in particular the plateau surface 53, in radial direction. The circumferential outer face 58 also constitutes a section of the cap 14, which is described later in detail.

The ring-shaped circumferential wall 54 with its upper edge or free end 56 can also be considered as part of the trough 45. In detail, the inner face 57 confines the interior of the trough 45, while the circumferential outer face 58 forms the outer contour of the trough 45, which is in fact the outer surface of the cap 14. The ring-shaped circumferential wall 54 thus constitutes an upper part of the trough 45.

FIGS. 4 to 6 provide detailed views of the growth medium support plate 12.

The growth medium support plate 12 comprises a microbiological testing area 60. Typically, the microbiological testing area 60 is coated with a growth medium 62. For clarity reasons, the growth medium 62 is omitted in most of the presented figures to allow a more detailed view on the microbiological testing area 60.

The microbiological testing area 60 can be located on either the first side 22 or the second side 24. More preferably, the microbiological testing area 60 is located on both sides 22, 24. As described above, each of the sides 22, 24 has a plane and even surface 25, on which the microbiological testing area 60 can be located.

The microbiological testing area 60 extends between the free end 18 of the distal portion 16 and the proximal portion 20 of the growth medium support plate 12.

Further, the microbiological testing area 60 is defined by side walls 64 protruding from the growth medium support plate 12. In detail, the side walls 64 are configured to confine a rectangular recess 66. In other words, the side walls 64 are circumferentially framing the recess 66 in a rectangular manner. The recess 66 further comprises a bottom surface 68 corresponding to the flat and even surface 25.

The microbiological testing area 60 also comprises a plurality of inner walls 70 or separating walls protruding from the bottom surface 68, which are crossing each other at a 90° angle thereby separating the recess 66 into a plurality of subareas 71. As shown in the embodiment of FIGS. 1 to 6, the microbiological testing area 60 comprises ten subareas 71. In the embodiment shown, the plurality of inner walls 70 consists of two kinds of inner walls 70, one wall arranged in parallel to the axial direction A D of the growth medium support plate 12 and the other wall arranged perpendicular to the axial direction A_{D} of the growth medium support plate 12 such that a chessboard-like pattern is formed. Therefore, each of the plurality of subareas 71 has a regular-polygonal shape that is defined by the inner walls 70 and the side walls 64. In the embodiment of FIGS. 1 to 6, each of the ten subareas 71 has a square-like shape.

Other arrangements of the subareas 71 are possible. For example, the inner walls 70 can be arranged in a triangular, pentagonal or hexagonal shape. Moreover, the number of subareas 71 in the microbiological testing area 60 can vary, depending on the size of the growth medium support plate 12 and the number of tests required. A minimum of about 4 subareas 71 is preferred from a practical point of view to minimize material consumption.

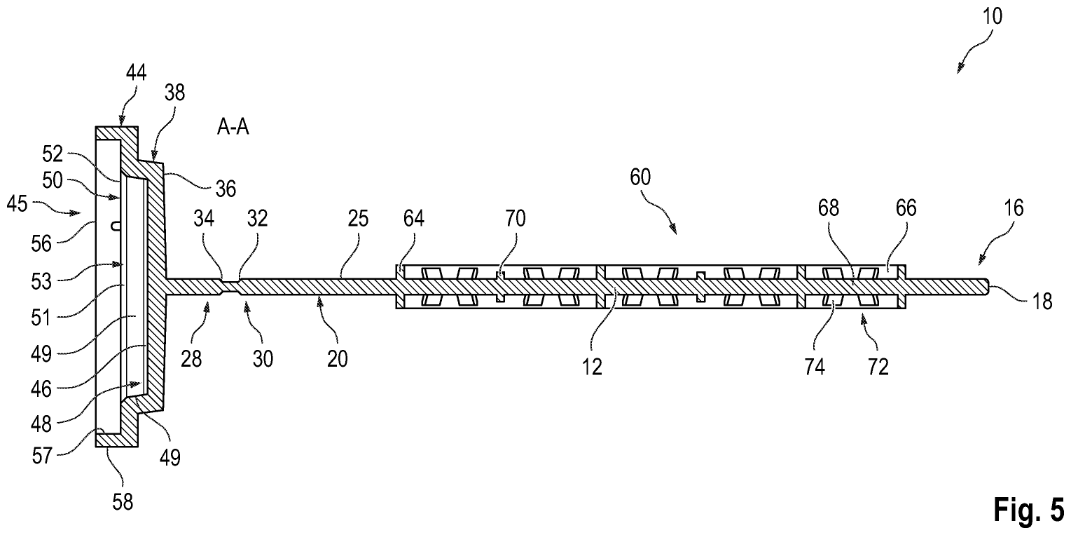

As shown in FIG. 5, the inner walls 70 have a smaller height compared to the side walls 64. The height is measured from the bottom surface 68 to the edge of the inner walls 70 or the side wall 64, respectively. Both, the side walls 64 and the inner walls 70, extend from the bottom surface 68 orthogonally upwards to a free end or upper edge. The side walls 64 may further comprise a beveled section 69 that extends from the bottom surface 68 into a straight part of the side wall 64.

Each of the plurality of subareas 71 comprises one anchoring unit 72.

In the embodiment shown in FIGS. 1 to 6, each anchoring unit 72 consists of four anchoring elements 74, which are symmetrically arranged to each other on the bottom surface 68. According to this embodiment, the four anchoring elements 74 are symmetrically linked to each other by an n-fold of the rotation axis R₉₀ oriented perpendicular to a plane defined by the growth medium support plate 12 or bottom surface 68. The integer “n” denotes the number of anchoring elements 74. In the present embodiment, n is equal to 4. Further, the rotation axis R₉₀ falls within the center of the square-like subareas 71. The rotation axis R₉₀ transfers the anchoring elements 74 into each other by a 90° rotation.

According to another embodiment (not shown), the arrangement of anchoring elements 74 of one anchoring unit 72 can be arbitrary. Thus, the anchoring elements 74 can be randomly distributed on the bottom surface 68.

The number of the anchoring elements 74 within one anchoring unit 72 may vary. Preferably, one anchoring unit 72 may have 2, 3, 4, 5, 6 or more than 6 anchoring elements 74.

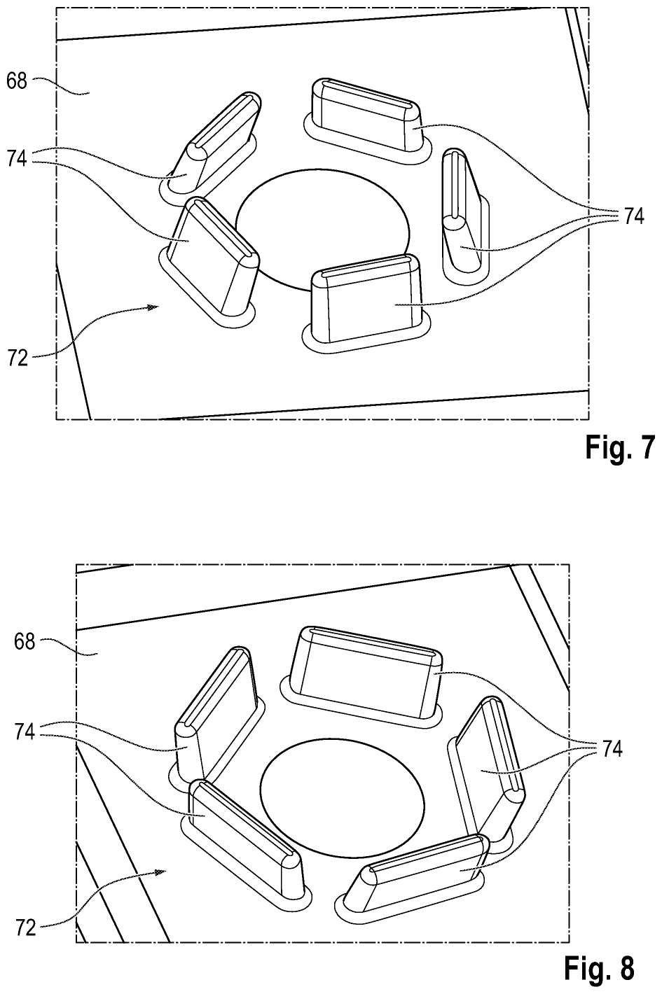

As shown in FIGS. 7 to 10, the arrangement of anchoring elements 74 inside one anchoring unit 72 may vary. In FIGS. 7 and 8, in total, six anchoring elements are arranged in a hexagonal fashion. While in FIG. 8 the undercuts are arranged opposite to each other, in FIG. 8 the undercuts are oriented in an opposing direction to each other.

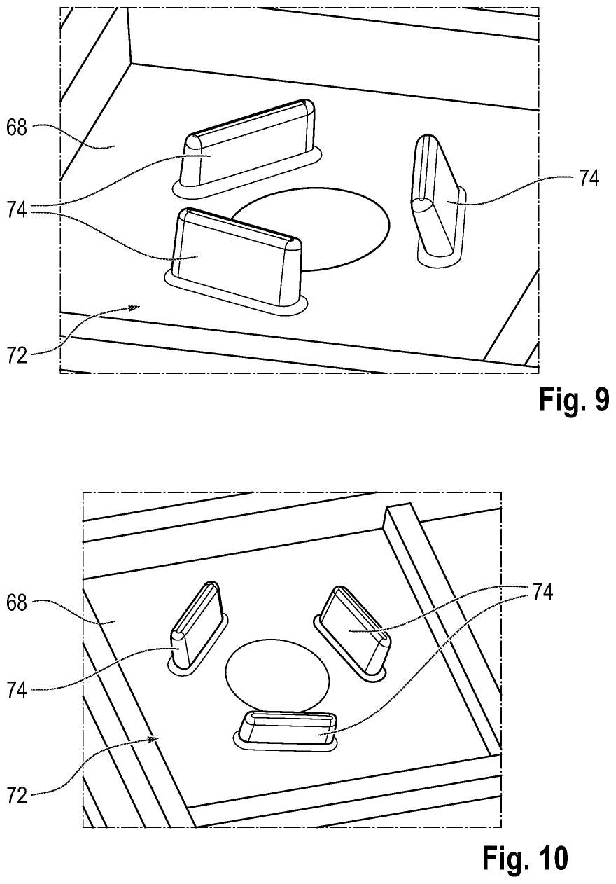

The same applies to the FIGS. 9 and 10 with the exception that merely three anchoring elements 74 are arranged within one anchoring unit 72. Here, the anchoring elements 74 have trigonal symmetry.

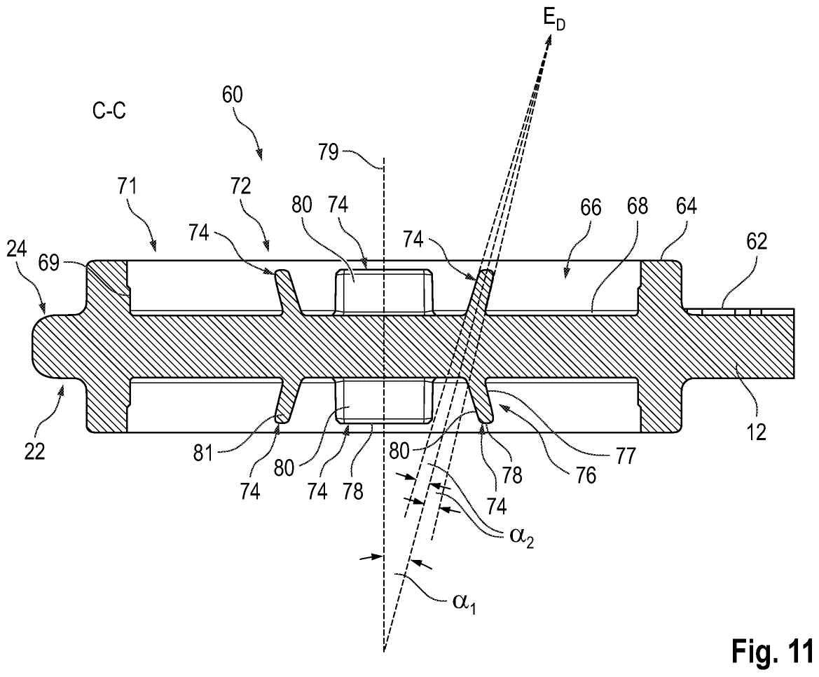

In the following, an anchoring element 74 is discussed in detail (see FIG. 11). The anchoring element 74 has a fin-like shape and protrudes from the bottom surface 68. Furthermore, the anchoring element 74 is inclined with respect to a plane 79 perpendicular to the bottom surface 68 to form an undercut 76 between the bottom surface 68 and the anchoring element 74. An extension direction ED of the anchoring element 74 from the bottom surface 68 is also shown in FIG. 11.

The undercut 76 is attributed to an undercut side 77 of the anchoring element 74. The undercut side 77 is also inclined along the extension direction ED of the anchoring element 74 and faces towards the bottom surface 68. The anchoring element 74 further comprises an inner side 80 arranged opposite to the undercut side 77 and facing adjacent anchoring elements 74. The inner side 80 and the undercut side 77 constitute the longitudinal sides of the fin-like anchoring element 74. The inner side 80 and the undercut side 77 are connected by two lateral sides 81 forming a front of the anchoring element. The two lateral sides 81 are arranged on opposite ends of the anchoring element. The top of the anchoring element 74 is formed by the free end 78.

The anchoring element 74 thus comprises an inner side 80, an undercut side 77, two lateral sides 81 and a free end 78 end providing a fin-like structure.

FIG. 11 shows a cross-section through the anchoring element 74 having the above-described fin-like structure. An angle α₁ between an extension direction ED of the anchoring element 74, also denoted as the inclined direction of the anchoring element 74, and the plane 79 perpendicular to the bottom surface 68 is about 15°. Further, an angle α₂ between the extension direction E_{D} of the free end 78 of the anchoring element 74 and the undercut side 77 or the inner side 80, respectively, is in the range of about 2°. In other words, the inner side 80 and the undercut side 77 may not be aligned parallel to each other. Rather, the anchoring element 74 is tapered by means of an opening angle between the inner side 80 and the undercut side 77 of 2×α₂ towards the bottom surface 68.

Variations of the described embodiment are possible. In particular, the angle α₁ may vary between 5° and 45°. The structure of the anchoring element may be varied by providing pins, ribs, rods, cones, ridges, polyhedrons and plates, or any other suitable shape having an undercut.

Further, FIG. 11 shows that the height of the free end 78 of the anchoring element 74 may be higher as compared to the height of the inner walls 70 but is lower compared to the height of side walls 64.

In the shown embodiment, the anchoring unit 72 comprises four of the anchoring elements 74 described above. As shown in FIGS. 1 to 6, the inner sides 80 of the anchoring elements 74 arranged within one anchoring unit 72 are each facing towards the center of the subarea 71. Vice versa, each of the undercut sides 77 of the anchoring elements 74 faces away from the center of the subarea 71.

The microbiological testing device 10 including the cap 14 and the growth medium support plate 12 is made as one-piece, preferably wherein said one-piece is injection molded from a thermoplastic polymer selected from the group consisting of high-density polyethylene and low-density polyethylene and combinations thereof. Further, the microbiological testing device comprising the cap and the support plate can be produced in a one-step process. As it is exemplary shown in FIGS. 7 to 10, the center of the subarea 71 can have an ejector pin pad, which are leftover marks from ejector pins used in the injection molding process.

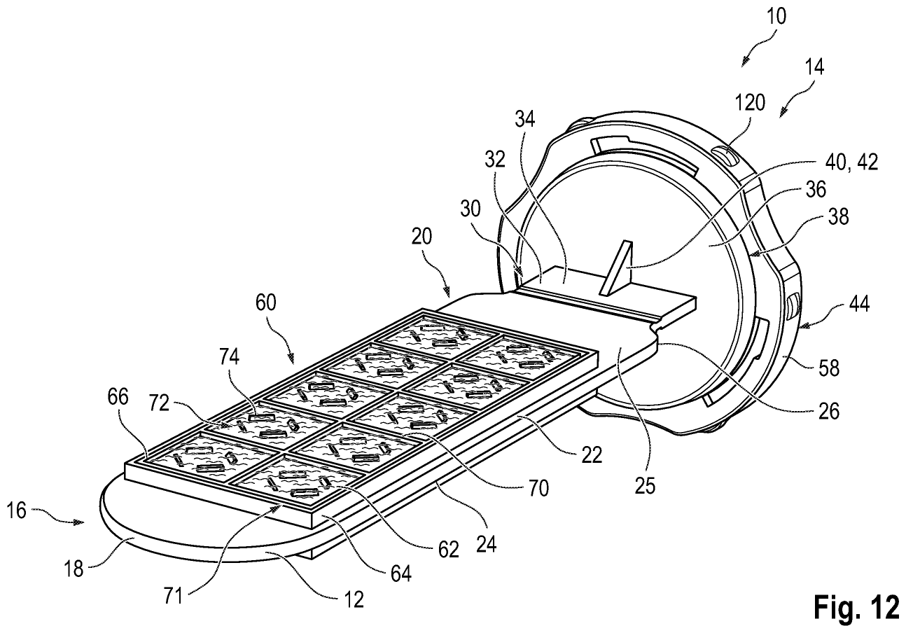

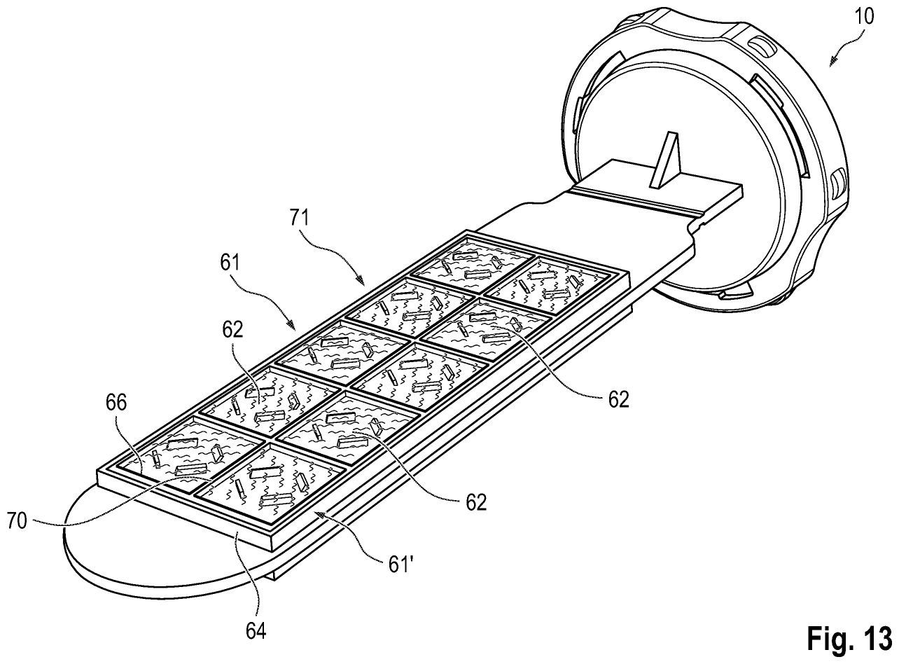

As described above, the microbiological growth medium is coated with a growth medium 62 as show in FIGS. 12 and 13. In particular, the recess 66 of the microbiological testing area 60 is filled with the growth medium 62 such that the growth medium 62 completely covers the anchoring elements 74 and the anchoring units 72. The growth medium 62 is typically a culture medium suitable for the growth of microorganisms such as agar. In the embodiment shown in FIG. 12, the growth medium 62 also covers the inner walls 70 and reaches up to the upper edges of the side walls 64. In other words, the microbiological testing area 60 comprises one single and continuous culture medium 62.

In another embodiment shown FIG. 13, the microbiological testing area 60 comprises a plurality of discrete fields 61, each being composed of culture medium 62, wherein each discrete field 61 of each of the plurality of discrete fields 61 is disposed each in a recess 66 of the plurality of recesses (66). As it can be seen in FIG. 13, the discrete fields 61 correspond to the sub-areas 71 defined by inner walls 70 and the side walls 64, wherein the inner walls 70 are separating the discrete field 61 from each other. This can, for example, be realized by increasing the height of the inner walls 70 e.g., as high or higher than the side walls 64 or by filling a reduced amount of culture medium in each of the plurality of recesses 66 thereby creating discrete fields 61.

As it is indicated in FIG. 13, it is preferred that at least two of the plurality of discrete fields (61, 61′) of culture media 62 have a different formulation. It might also be that the culture medium 62 of each discrete field 61 of the plurality of fields has a formulation being different from the formulations of each of the other of the plurality of discrete fields 61. For example, in FIG. 13 two different formulations are used for the culture medium 62 resulting in a chessboard-like pattern composed of two kinds of discrete fields (61, 61′) in the microbiological testing area 60. Each kind of discrete field (61, 61′) may have its own formulation for detecting a different sort of microorganism.

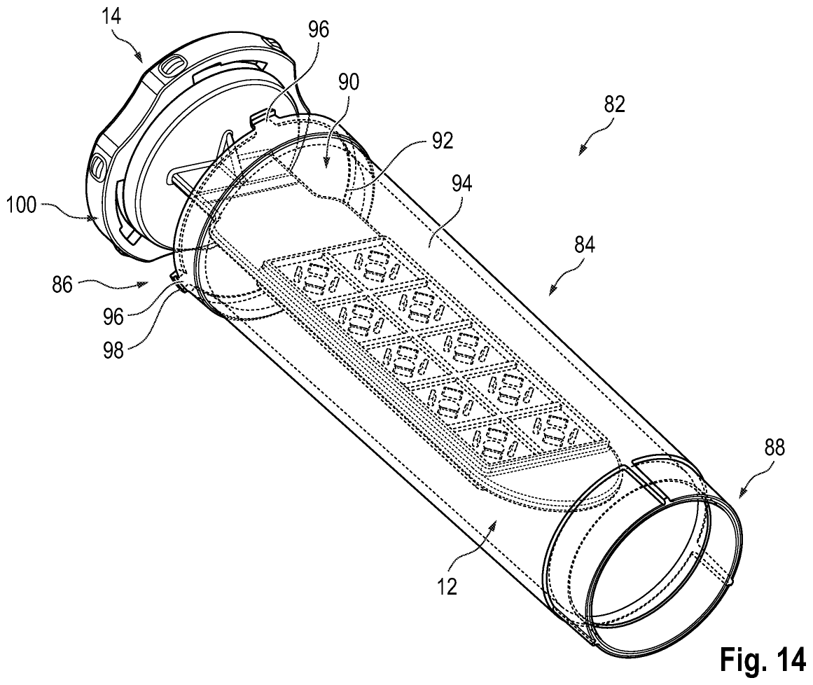

FIG. 14 depicts an isometric view of a microbiological testing assembly 82 comprising a microbiological testing device 10 and a vial 84 for receiving the microbiological testing device 10.

The vial 84 defines a housing having an open end 86 and closed end 88 and an interior 90 located in between.

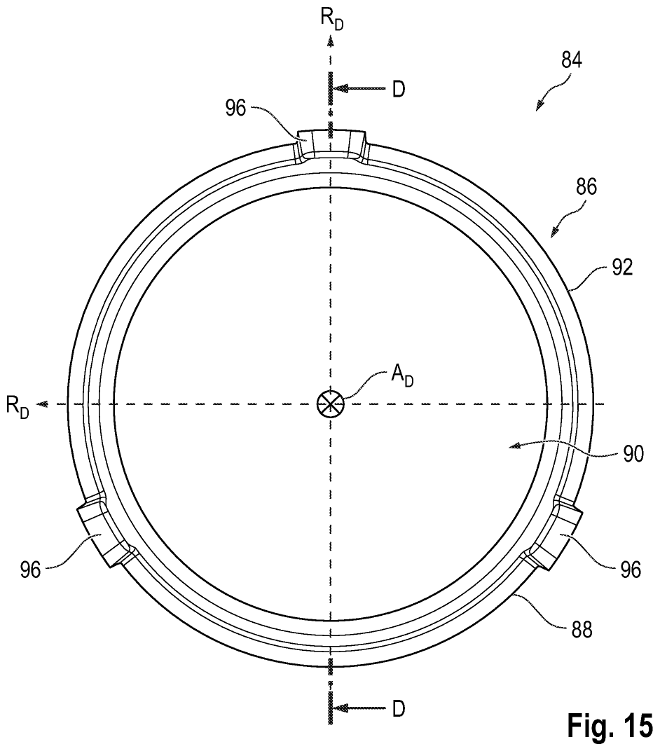

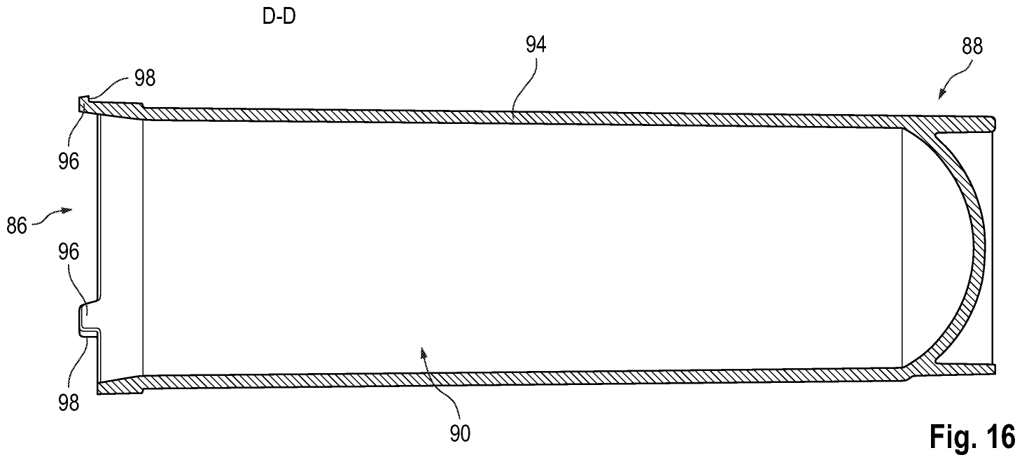

As can be seen from FIGS. 15 and 16, the vial 84 has a cylindrical elongated shape, which extends in axial direction A_{D}. Said open end 86 comprises an opening 92, which has a circular shape and is adapted to receive the growth medium support plate 12, and engage with the abutment portion 44 of the cap 14, such that the growth medium support plate 12 can be completely encapsulated in the interior 90 of the vial 84.

The vial 84 is made from a translucent material such that the interior 90 of the vial 84 can be seen from outside. Thus, the growth of bacteria or fungi can be traced by visual inspection through the transparent vial 84 during culturing on the growth medium 62. The vial 84 can be made by injection molding of clear polystyrene or translucent polyethylene.

The housing defined by the vial 84 comprises a cylindrical vessel wall 94 extending between the opening 92 and the closed end 88 that circumferentially encloses the interior 90 in a radial direction.

As shown in FIGS. 15 and 16, the vial 84 comprises three pins 96 that are equally distributed along an upper edge of the opening 92 in circumferential direction. In other words, each of the pins 96 is located on a circular line with a 120° angle between each of the pins 96. Furthermore, each of the pins 96 comprises a protrusion, which extends radially outwards from the opening 92. This protrusion is also denoted in the following as bayonet interlocking element 98.

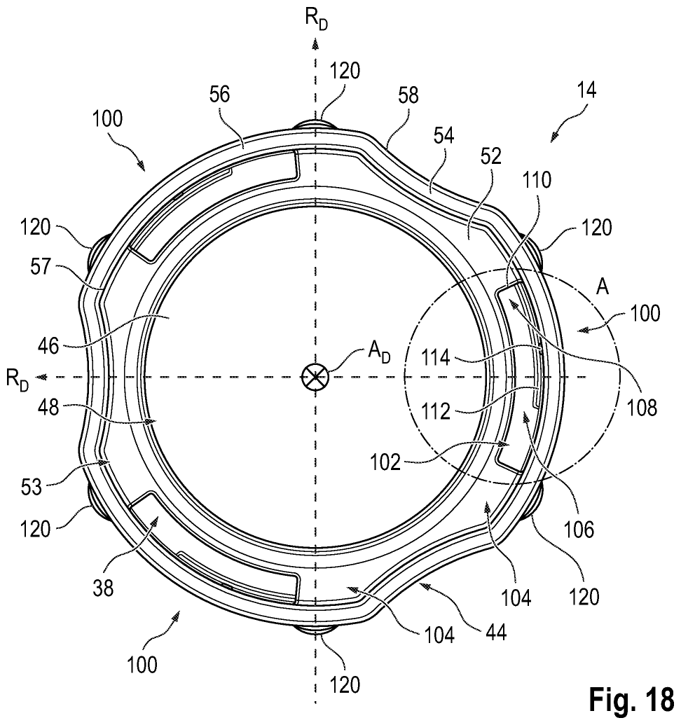

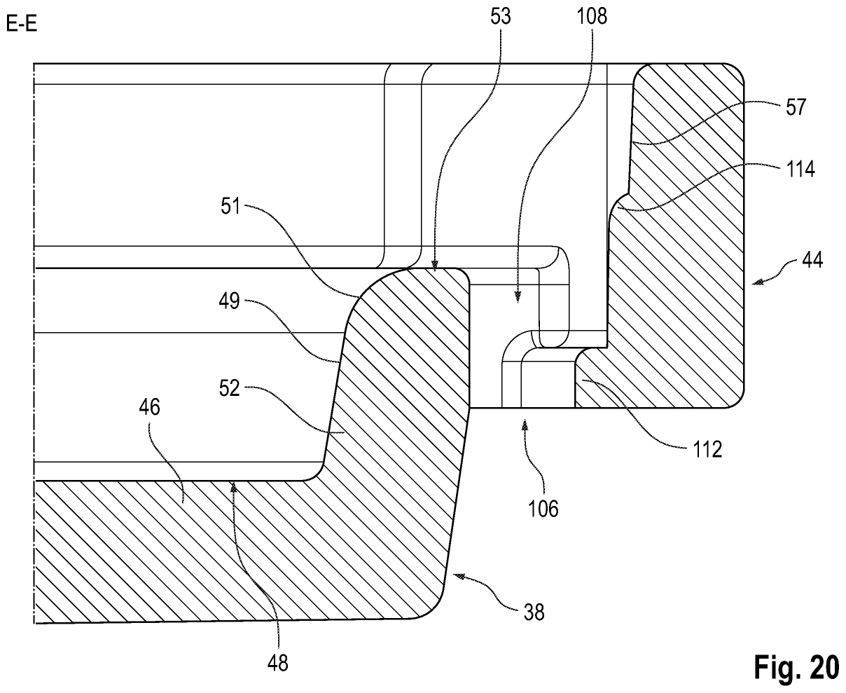

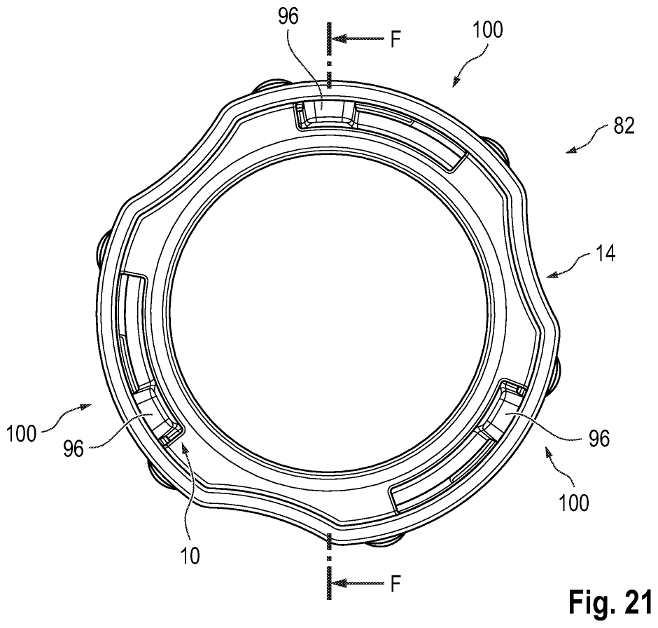

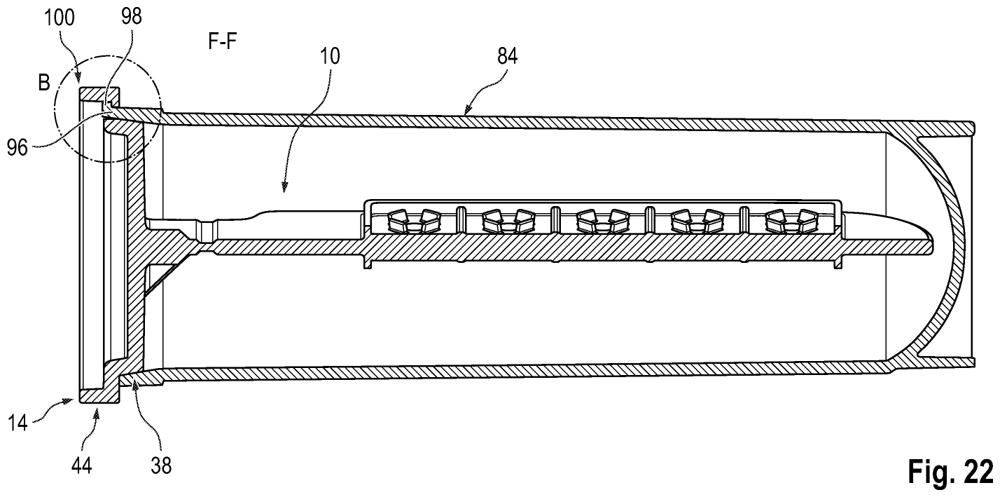

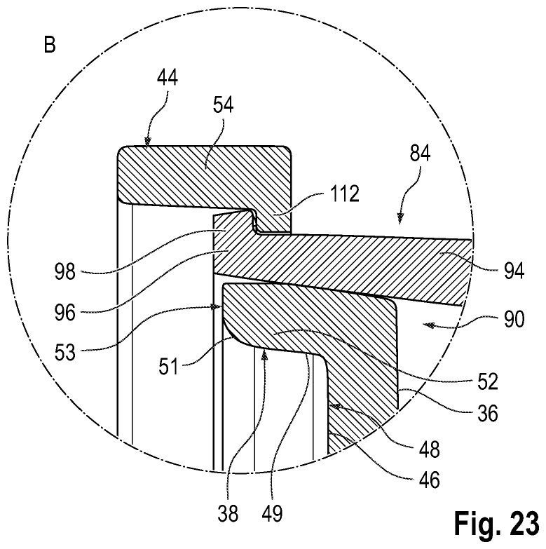

As depicted in FIGS. 12 to 23, the opening 92 of the vial 84 is adapted to engage with the cap 14 of the microbiological testing assembly 82. For this reason, the cap 14 is equipped with a bayonet fastening 100. The bayonet fastening 100 is adapted to engage with the opening 92 of the vial 84 for fastening the vial 84 to the cap 14.

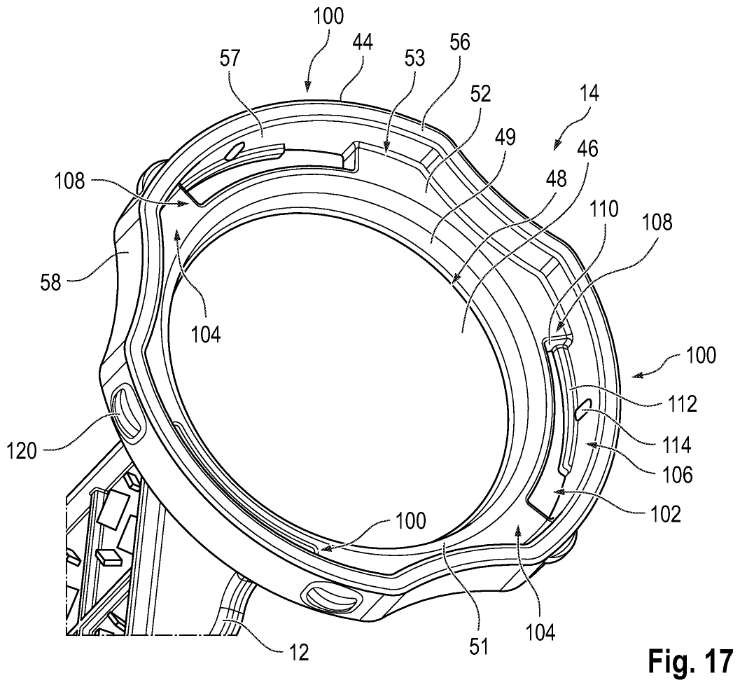

The bayonet fastening 100 comprises at least one receptor opening 102 extending through a border portion 104 of the cap 14 in axial direction A_{D}. In particular, the border portion 104 comprising the receptor opening 102 corresponds to the abutment portion 44 and constitutes the outer part of the above-described plateau surface 53 of the inner stage 52 (FIG. 17). In other words, the abutment portion 44 comprises a tunnel in the form of the receptor opening 102 axially extending through the inner stage 52. In general, the receptor opening 102 has an essentially rectangular shape.

The bayonet fastening 100 further comprises a slot portion 106 having the form of a slit that extends from the receptor opening 102 to a bearing portion 108 in circumferential direction of the disk-shaped bottom plate 38.

The slot portion 106 has a curved path, respectively a curved shape, having the same curvature as the inner face 57 of the ring-shaped circumferential wall 54 and the inner stage 52, which both limit an extension of the slot portion 106 in radial direction. The extension in circumferential direction is limited by bearing portion 108 and the receptor opening 102. Further, the bearing portion comprises a dead end 110.

The slot portion 106 further comprises a bayonet rail 112. The bayonet rail 112 is formed as a protrusion of the circumferential ring-shaped wall 54 directed radially inwards and having a free end extending towards the inner stage 52 and into the slot portion 106. Further, the bayonet rail 112 is inclined in axial direction A_{D}. The bayonet rail 112 can therefore also be seen as a ramp with an inclined direction towards the plateau surface 53.

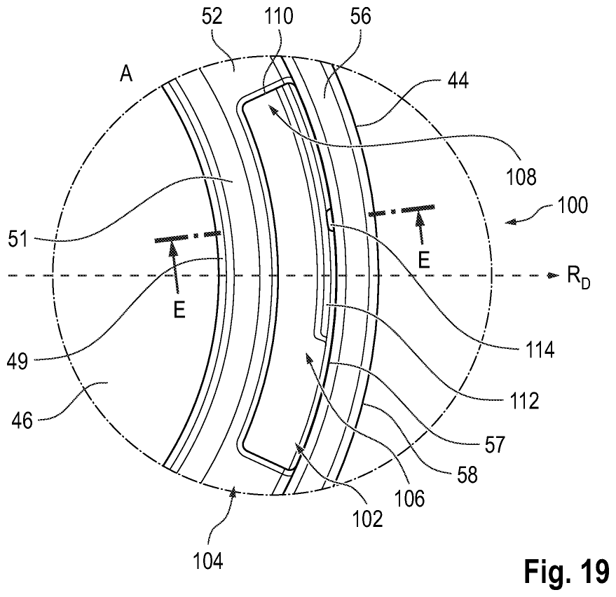

Further, the bayonet fastening 100 comprises a bayonet-latching element 114 that is located adjacent to the bearing portion 108. The bayonet-latching element 114 is attached to the inner face 57 of the circumferential ring-shaped wall 54 and has a free end extending towards the inner stage 52 and into the slot portion 106. Further, the bayonet-latching element 114 is located in axial direction A_{D} above the bayonet rail 112. As shown in FIG. 19, the bayonet-latching element 114 is constructed as a two-part element, wherein an empty space is located in between each of the two elements such that the two elements together can be elastically compressed against each other. However, the bayonet-latching element 114 does not extend beyond the bayonet rail 112.

In the embodiment shown, the cap 14 comprises three of the above discussed bayonet fastenings 100, wherein each of the bayonet fastenings 100 is located on a circular line with a 120° angle between each of the bayonet fastenings 100.

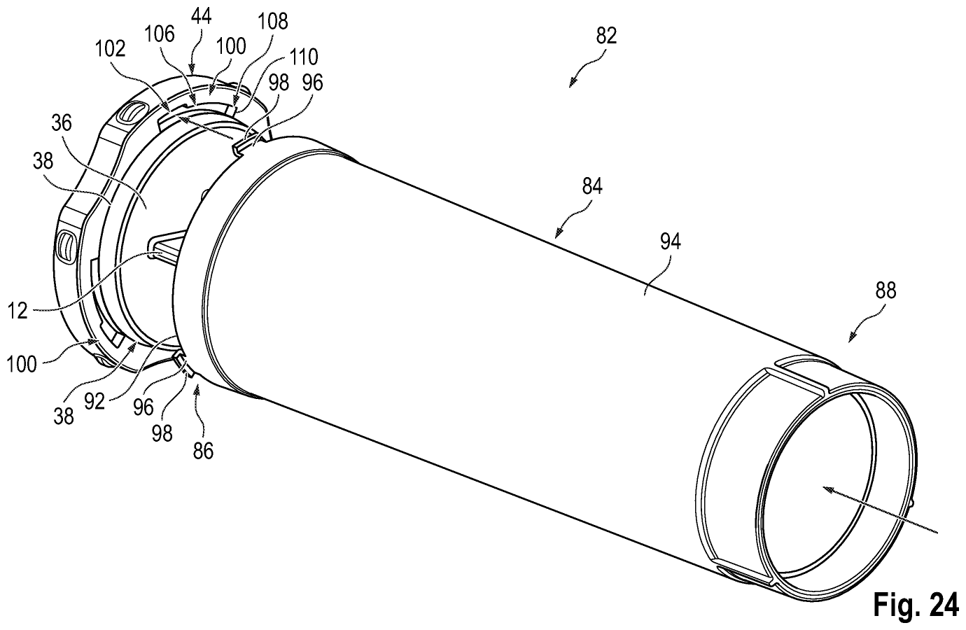

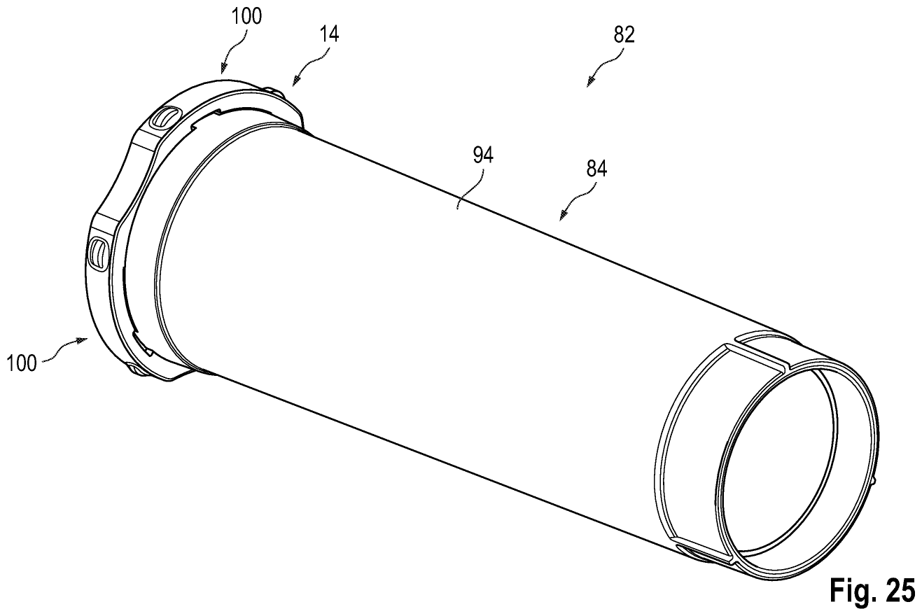

The interlocking mechanism, respectively the fastening mechanism, of the bayonet fastening 100, designed to fasten the cap 14 to the vial 84 is shown in FIGS. 24 to 27.

In the following, the fastening mechanism of the bayonet fastening 100 is explained in detail. The vial 84 can be moved in an axial direction A D towards the bottom side 36 of the cap 14 such that each of the pins 96 is inserted into one of the receptor openings 102 of each of the bayonet fastenings 100. Thus, each of the pins 96 fits into one receptor opening 102. Subsequently, the microbiological testing device 10 and the vial 84 are twisted against each other such that each of the bayonet interlocking elements 98 of the pins 96 slides upwards along each of the bayonet rails 112 of the slot portions 106 into the bearing portions 108 against the dead ends 110. While this movement occurs, each of the interlocking elements 98 of the pins 96 slides over one of the bayonet-latching elements 114. In this respect, each of the bayonet-latching elements 114 provides a mechanical resistance which the bayonet interlocking element 98 has to overcome in order to slide into the bearing portion 108. After sliding over the bayonet-latching element 114, each of the pins 96 has reached its end position in the bearing portion 108. The microbiological testing assembly 82 is then in a locked state.

The end position of each of the pins 96 is locked by each of the bayonet-latching elements 114 to prevent an accidental movement of the pins 96. The vial 84 cannot be removed from the cap 14 without applying a certain amount of force to have the bayonet interlocking elements 98 slide backwards, down on the bayonet rail 112 and over the bayonet-latching element 114. If the bayonet interlocking elements 98 slide over the bayonet-latching element 114 back into the receptor opening 102 again, the vial 84 can be retrieved from the cap 14 by pulling the pins 96 out of the receptor openings 102. The microbiological testing assembly 82 then is in an unlocked state.

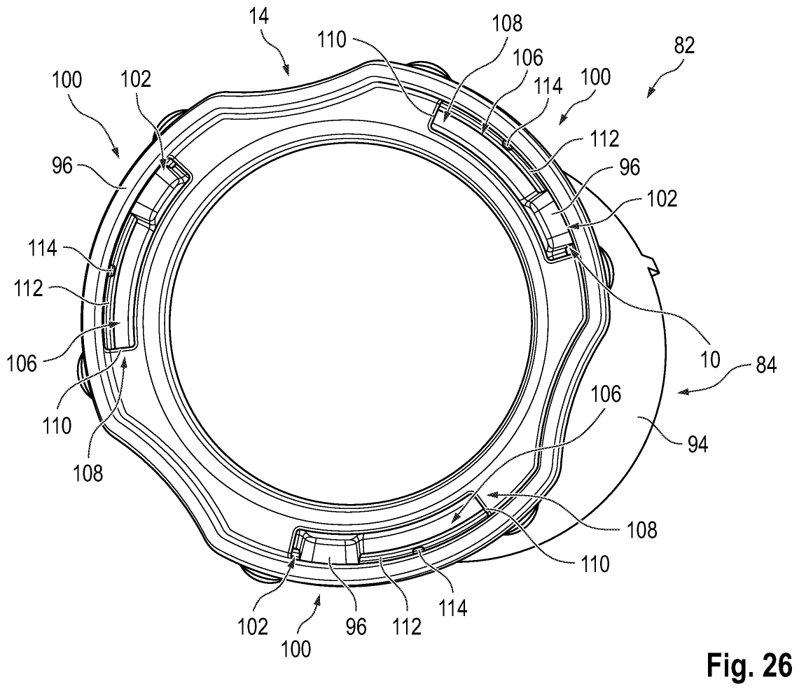

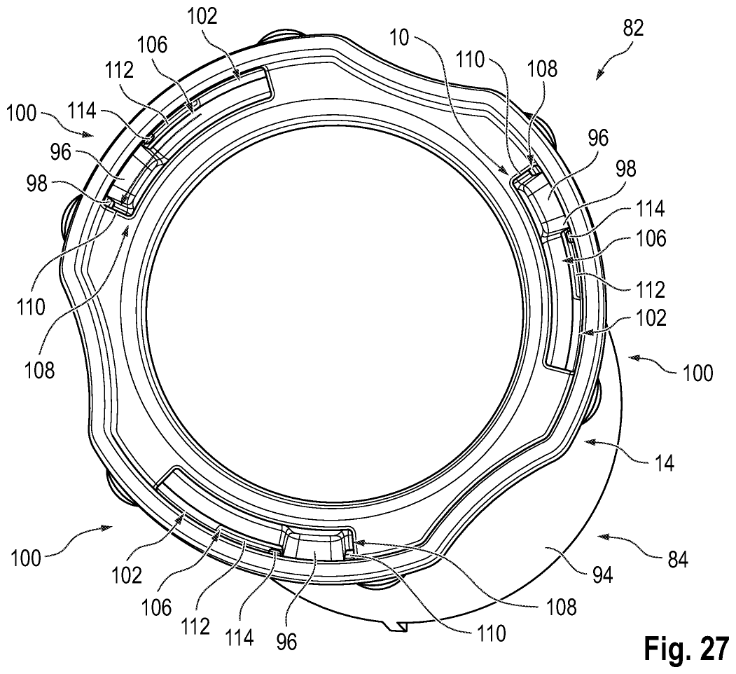

In order to enable a smooth sliding of the bayonet interlocking element 98 along the bayonet rail 112, the bayonet rail 112 and the bayonet interlocking element 98 have corresponding protrusions in radial direction R_{D}. In other words, the bayonet rail 112 has a protrusion directed radially inwards, which corresponds in terms of the extension direction to the protrusion of the bayonet interlocking element 98 directed radially outwards. The sliding of the bayonet interlocking element 98 along the bayonet rail 112 is also shown in detail in FIGS. 26 and 27.

The mechanical resistance that has to be overcome by applying a certain amount of force when twisting the cap 14 and the microbiological testing device 10 against each other can be adjusted by means of the radially inwards-directed extension of the bayonet-latching element 114. If the bayonet-latching element 114 has a small protrusion extending radially inwards, the force to be applied for sliding the pin 96 into the bearing position 108 is rather low. If the protrusion extending radially inwards is rather big, the force that needs to be applied to lock or fasten the cap 14 to the vial 84 is higher.

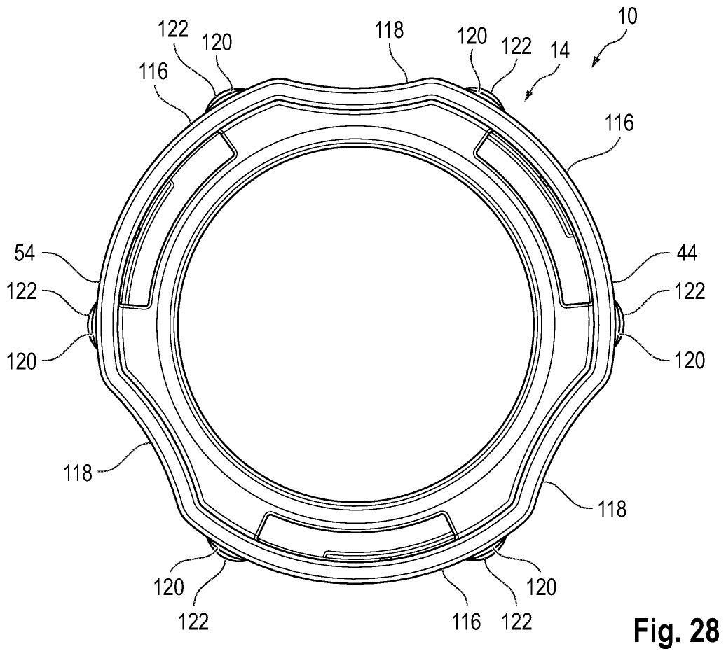

FIG. 28 illustrates the cap 14 for a microbiological testing device. The cap 14 may have some or all of the features described above.

The cap 14 has curved convex 116 and concave 118 sections, which are alternately arranged in a circumferential direction of the cap 14. In particular, the convex and concave curved sections 116, 118 are part of the trough. More specifically, the circumferential outer face 58 of the ring-shaped wall 54 of trough 45 is configured to comprise three convex curved sections 116 and three concave curved sections 118 such that the cap 14 seen from an axial direction A_{D} has a quasi-hexagonal shape.

Further, at least one of the alternately arranged convex and concave curved sections 116, 118 may comprise at least one anti-twist element 120.

The anti-twist element 120 has a free end 122 pointing radially outwards. In particular, the anti-twist element 120 is located at the circumferential outer face 58 of the ring-shaped wall 54.

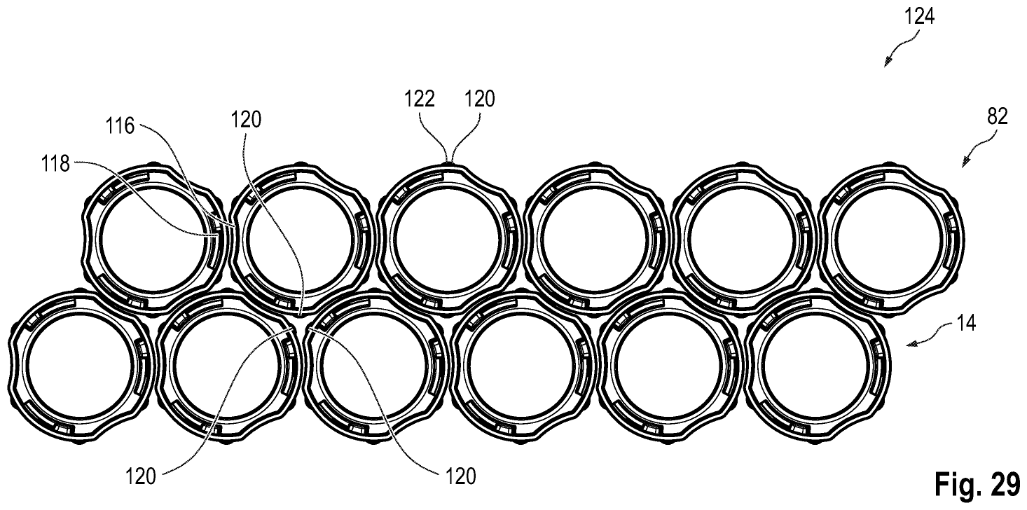

The advantage of providing a cap having a quasi-hexagonal shape is illustrated in FIG. 29. FIG. 29 depicts a top view of a package 124 of a plurality of microbiological testing assemblies 82. The microbiological testing assemblies 82 are arranged in a plane of a quasi-close packing of equal spheres enabling a high packing density of the plurality of microbiological testing assemblies 82.

The quasi-hexagonal shape of the cap 14 allows that each of the convex curved sections 116 of one microbiological testing assembly 82 can engage with a concave curved section 118 of another microbiological testing assembly 82 such that each of the microbiological testing assemblies 82 is arranged non-rotatably inside the package 124. As shown in FIG. 29, up to six neighboring microbiological testing assemblies 82 surround one microbiological testing assembly 82 in plane. Thus, the convex and concave curved sections 116, 118 can interlock into the convex and concave curved sections 116, 118 of the other adjacent microbiological testing assemblies 82. Therefore, not only is the microbiological testing assembly 82 non-rotatably immobilized, but rather the whole package 124 is non-rotatably locked.

The anti-twisting effect of the convex and concave curved sections 116, 118 is further enhanced by the anti-twist element 120, into which the convex and concave sections 116, 118 can hook in.

Although the invention has been described in conjunction with specific embodiments thereof, it is evident that many alternatives, modifications and variations will be apparent to those skilled in the art. For example, all of the above-described embodiments can be combined with each other, or can be used separately and independently from each other. The scope of the invention is defined by the claims which follow.

1. A microbiological testing device comprising:
a growth medium support plate and a cap attached to the growth medium support plate,
the growth medium support plate comprising a microbiological testing area extending between a distal portion having a free end and a proximal portion attached to the cap,
wherein the microbiological testing area comprises at least one recess for receiving a microbiological growth medium,
wherein the least one recess comprises a bottom surface and at least one anchoring element protruding from the bottom surface, and
wherein the at least one anchoring element comprises an undercut for engaging with the microbiological growth medium.

2. The microbiological testing device according to claim 1, wherein the at least one anchoring element is inclined with respect to a plane perpendicular to the bottom surface to form the undercut between the bottom surface and the at least one anchoring element).

3. The microbiological testing device according to claim 2, wherein an angle α₁ between an inclined direction of the at least one anchoring element and the plane perpendicular to the bottom surface is in the range of 5 to 45°.

4. The microbiological testing device according to claim 1, wherein the at least one anchoring element has free end, and wherein the free end is tapered.

5. The microbiological testing device according to claim 1, wherein the at least one anchoring element is selected from the group consisting of fins, pins, ribs, rods, cones, ridges, polyhedrons and plates.

6. The microbiological testing device according to claim 1, wherein the growth medium support plate comprises a plurality of anchoring elements arranged on the bottom surface.

7. The microbiological testing device according to claim 6, wherein the plurality of anchoring elements form at least one anchoring unit on the bottom surface, wherein the at least one anchoring unit comprises 2, 3, 4, 5, 6 or more than 6 anchoring elements.

8. The microbiological testing device according to claim 7, wherein the undercuts of the anchoring elements of one anchoring unit are each arranged in opposite or in opposing orientation to each other.

9. The microbiological testing device according to claim 7, wherein the microbiological testing area comprises a plurality of recesses, wherein each recess of the plurality of recesses has a regular-polygonal shape that is defined by wall elements framing each recess of the plurality of recesses, wherein each recess of the plurality of recesses includes at least one anchoring unit.

10. The microbiological testing device according to claim 9, wherein the microbiological testing area comprises a plurality of discrete fields, each discrete field being composed of a culture medium, wherein each discrete field of the plurality of discrete fields is disposed in a recess of the plurality of recesses.

11. The microbiological testing device according to claim 10, wherein the culture medium of at least two discrete fields of the plurality of discrete fields has a different formulation.

12. The microbiological testing device according to claim 1, wherein the cap and the growth medium support plate are made as one-piece, preferably wherein said one-piece is injection molded from a thermoplastic polymer.

13. A microbiological testing assembly comprising a microbiological testing device and a vial for receiving the microbiological testing device,
the microbiological testing device comprising:
a growth medium support plate and a cap attached to the growth medium support plate, wherein the growth medium support plate comprising a microbiological testing area extending between a distal portion having a free end and a proximal portion attached to the cap, and
wherein the vial has an open end, a closed end and an interior located in-between, said open end comprises an opening that is adapted to receive the growth medium support plate such that the growth medium support plate can be completely encapsulated in the interior of the vial, and
wherein the cap comprises at least one bayonet fastening adapted to engage with the open end of the vial for fastening the vial to the cap.

14. The microbiological testing assembly according to claim 13, wherein the bayonet fastening comprises at least one receptor opening extending through an abutment portion of the cap in axial direction, wherein the bayonet fastening further comprises a slot portion that extends from the receptor opening to a bearing portion in circumferential direction of the cap.

15. The microbiological testing assembly according to claim 14, wherein the slot portion comprises a bayonet rail that has an inclination in axial direction and a bayonet-latching element located adjacent to the bearing portion,
wherein the vial comprises at least one pin located adjacent to the opening of the vial,
wherein the at least one pin comprises a bayonet interlocking element, and wherein the at least one pin is adapted to match into the receptor opening of the bayonet fastening and to slide upwards along the bayonet rail of the slot portion into the bearing portion, wherein an end position of the pin in the bearing portion is locked by the bayonet-latching element.

16. A cap for a microbiological testing device, comprising:
a disk-shaped bottom plate having a bottom side that is attachable to a growth medium support plate and an abutment portion located opposite to the bottom side and extending radially outward of the disk-shaped bottom plate forming the front side of the cap, and
wherein an outer diameter of the abutment portion is greater than an outer diameter of the disk-shaped bottom plate, wherein a shoulder is formed between the disk-shaped bottom plate and the abutment portion.

17. The cap-according to claim 16, wherein the disk-shaped bottom plate and the abutment portion-form a trough on the front side of the cap, opposite to the bottom side of the disk-shaped bottom plate.

18. The cap according to claim 17, wherein a bottom of the trough comprises a writable surface.

19. The cap according to claim 18, wherein the writable surface is circumferentially framed by a side wall of the trough.

20. The cap according to claim 16, wherein the cap is attached to the growth medium support plate forming a microbiological testing device, and wherein the cap comprises a writable surface.

21. The cap according to claim 20, wherein the cap and the growth medium support plate are made as one-piece.

22. The cap according to claim 16, wherein the cap comprises a circumferential outer face extending from the abutment portion, wherein the circumferential outer face has alternately arranged convex and concave curved sections.

23. The cap according to claim 22, wherein the circumferential outer face comprises three convex sections and three concave sections such that the cap seen from an axial direction has a quasi-hexagonal shape.

24. The cap according to claim 22, wherein at least one of the alternately arranged convex and concave curved sections comprises at least one anti-twist element, wherein the at least one anti-twist element extends from the at least one of the alternately arranged convex and concave curved sections radially outwards.
